(19) United States
(12) Patent Application Publication
(10) Pub. No.: US 2005/0214811 A1
(43) Pub. Date: Sep. 29, 2005

(54) Processing and managing genetic information

(76) Inventors: David M. Margulies, Newton, MA (US); Joseph A. Majzoub, Wellesley, MA (US); Isaac S. Kohane, Newton, MA (US); Joyce S. Samet, Brookline, MA (US)

Correspondence Address: FISH & RICHARDSON PC, P.O. BOX 1022, MINNEAPOLIS, MN, 55440-1022, US

(21) Appl. No.: 11/009,236

(22) Filed: Dec. 10, 2004

Related U.S. Application Data
(60) Provisional application No. 60/529,274, filed on Dec. 12, 2003.
(60) Provisional application No. 60/550,784, filed on Mar. 5, 2004.
(60) Provisional application No. 60/591,668, filed on Jul. 28, 2004.

(51) Int. Cl. C12Q 1/68; G06F 19/00; G01N 33/48; G01N 33/50

(52) U.S. Cl. 435/6; 702/20

(57) ABSTRACT

Changes in association between a genetic variant and a disorder can be used as a prompt to automatically revise the diagnosis based on the patient's genetic information. For example, revisions in levels of confidence of a curated database of variants can trigger sending an updated report to the clinician or patient.

### CROSS-REFERENCE TO RELATED APPLICATIONS

This application claims priority to U.S. Application Ser. No. 60/529,274, filed on 12 Dec. 2003, Ser. No. 60/550,784, filed Mar. 5, 2004, and Ser. No. 60/591,668, filed on 28 Jul. 2004, the contents of all of which are hereby incorporated by reference in their entireties.

### DESCRIPTION OF THE INVENTION

Advances in medicine and biotechnology have increased the amount of information that can be used by clinicians to diagnose and care for their patients. These advances include evolving information about how genetic variation informs the diagnosis of disease.

Individuals, e.g., individuals that present with one or more disease associated phenotypes known to be associated with genetic variation, can be tested to obtain information about their genetic composition. This information can be used to provide a diagnosis and to make a clinical decision. However, the pace of biomedical research generates an evolving source of information, as does the aggregation of genetic and phenotypic information. In one aspect, the invention features a method for diagnosing and periodically reporting the confidence level of the diagnosis using sequence information from a test subject. The interpretation of the results of such sequence information is updated, e.g., as warranted by subsequent changes in information regarding the level of confidence between the subject's sequence information and the diagnosis of the disorder. Changes in information can become available through the scientific literature and test performance, and other sources.

A disorder includes diseases and clinical syndromes, as well as deviations from normal health that do not rise to the level of a disease or clinical syndrome. A clinical syndrome is a disorder that presents with common signs, symptoms or complaints. A clinical syndrome can have a probabilistic or causal relationship with one or more variants of one or more genes. A disorder can be manifested by multiple phenotypes. The disorder can be caused by one or more factors, including genetic factors. Whether a particular genetic factor is a cause of the disorder can be determined with varying levels of confidence.

The method typically uses a database of variants. A “variant” is an allele of a gene. A database of variants can include, for example, entries for variants at a particular loci and/or variants for multiple loci (e.g., at least one variant for each of the multiple loci). For example, the database includes information about variants in one or more genes associated with the disorder and information associating each of the variants with a level of confidence in the association of the disorder. The database can also include one or more database entries that correlate a combination of variants and a clinical state.

Examples of variants include polymorphisms (e.g., single nucleotide polymorphisms) and mutations (e.g., one or more of a deletion of at least one nucleotide, an inversion, a translocation, or an insertion of at least one nucleotide). Variants can be identified, for example, by comparing the sequence information for a subject to a reference sequence.

In one embodiment, the method includes determining the sequence of a target region of a gene in a subject, e.g., by sequencing the gene(s), or at least obtaining a partial sequence of one or more genes or by otherwise determining the identity of the one or more nucleotides in the target region. Determining a sequence can include any type of sequencing, e.g., Maxam-Gilbert sequencing, Sanger sequencing, ligase chain reaction, an inferential method, or any other method described herein. A “target region” is one or more nucleotides. The nucleotides may be contiguous or not contiguous.

The sequenced genes can be genes associated with the disorder, thereby providing sequence information for each test subject. The target region of the gene can include, e.g., at least a portion of a coding region, a portion of a regulatory region (e.g., a transcriptional or translational control region), or a portion of an intron.

The method can include storing sequence information in a database, e.g., a database that associates an identifier for each subject and the sequence information obtained from each test subject. The method can also include associating this sequence information with clinical information, e.g., clinical information that is also stored in the database. Examples of clinical information include: codified clinical annotations, phenotype information, and family history. The method can include: obtaining clinical information (e.g., a clinical annotation data set) about the test subject prior to or at the time of requisition for genetic testing.

The method can further include obtaining phenotypic or clinical information from one or more of the subjects, e.g., a parameter that indicates levels of a metabolite, e.g., a sugar or lipid metabolite, e.g., cholesterol, e.g., LDL or HDL particles, a parameter relating to other blood work, a physiological parameter (e.g., blood pressure, weight, etc.). Examples of phenotypes include an observable or measurable trait, which is heritable and includes heritable clinical information or parameters. Other examples of phenotypes include traits that are not heritable.

It is also possible to store an indicator that represents whether a subject requests an updated report for his/her genetic information.

The method can provide a first report for each test subject. The first report can include one or more of: information about the subject sequence, information as to whether the subject has the disorder, and information about the level of confidence in the diagnosis of the disorder. Information for first report can be produced by identifying those variants in the database of variants that are found in the respective subject's sequence information. The report can also include information about state of the database, e.g., at the time that the report was generated.

The method can also include sequencing the gene(s) in a subsequent subject, e.g., a subject whose genetic information is not yet entered into the database. The assessment of the subsequent subject can be informed by the evaluation of prior subject, particularly from associations arising from genetic and phenotypic information about the prior subjects. The assessment of the prior subject can also be informed by the evaluation of the subsequent subject. The report can also include information about the current state of the database, e.g., number of test subjects, total number of test subjects having the same variant, date of last update to the database, etc.

The method can include modifying the database, e.g., by (i) modifying the database of variants based on information about the subsequent subject; or (ii) modifying the database of variants based on information about the genes relevant to the disorder. For example, the information can be new information, e.g., from public or private electronic and paper sources. Other sources of information include compedia of gene variants and their associated clinical findings. Modification of the database can also include altering at least one association between a variant and a disorder (e.g., modifying the level of confidence in the diagnosis of the disorder), adding at least one association between a variant and a disorder, and adding a new variant that was absent from the database prior to the modifying. Modification of the database can include determining the sequence of the target region of the gene in a second or subsequent subject; and modifying the database of variants based on information about the second subject or any subsequent subject.

The method can further include preparing a second or subsequent report for one or more of the subjects, e.g., subjects whose first or prior report would be altered by the database modification or occurring as a result of (i) or (ii). The second or subsequent report typically includes information about the disorder, e.g., as determined by identifying those variants in the modified database of variants that are found in the subject's sequence information.

In one embodiment, the sequence information used for providing the second or subsequent report includes the sequence information obtained from the subject in conjunction with the issuance of the first report or includes information obtained prior to generation of the first report. A second report can be provided if no change is detected, and/or if (e.g., only if) a change is detected. The change can be a change in the level of confidence of the diagnosis.

In one embodiment, the second or subsequent report includes information about the level of confidence in the diagnosis of the disorder. The level of confidence in the second or subsequent report can be revised relative to a previous report. For example, the second report or subsequent report indicates a different level of confidence in the diagnosis of the disorder from that indicated in a corresponding first or previous report or that the level of confidence in the diagnosis is unchanged compared with the first or previous report.

The second report can indicate the same or a different diagnosis than the corresponding first report. This method can be repeated, e.g., to produce a third report and/or fourth report, etc. The second or subsequent report can provide an updated interpretation of the prior report to reflect changes in the knowledge of the level of confidence between the subject's variant(s) and the diagnosis of the disorder. A physician can use the first, second or subsequent report to determine whether to deliver or withhold a selected treatment (e.g., drug or surgical intervention) or to make a decision with regard to the management of the patient's care.

In one embodiment, identifying variants includes a step of comparing the sequence information for a subject to a reference sequence.

In one embodiment, the database of variants includes one or more records that correlate a combination of variants and a diagnosis of a clinical state, e.g., disorder.

In one embodiment, the database provides one or more of: a probability of disease association, a mode of inheritance, and presence or absence of specifically codified clinical findings. In one embodiment, the database provides information about clinical presentation for each variant.

The method can include other features described herein.

In one aspect, the invention features a method of storing genetic information obtained from testing. The method includes storing, in a first database, genetic information for an individual in association with a key, e.g., a key that does not recognizably describe the individual; storing the key, e.g., with information that identifies the individual in a second database; and enabling a third party to access information in the first database, but not the second database. For example, the keys are semantic free keys. For example, the database can include genetic information, diagnostic information, and/or pharmacological information.

The method can include other features described herein.

In one aspect, the invention features a method that includes: automatically detecting changes in a database that comprises records that associate genes or regions thereof with phenotypic information; optionally, generating an alert; producing a rule based on a change detected in the database; evaluating genetic information for multiple individuals using the rule; and generating a report that comprises results of the evaluation of at least one individual.

The method can further include updating the phenotypic database or making a decision, e.g., whether notification or a new report is required. The method can further include sending such notification or report. The method can include other features described herein.

In another aspect, the invention features a method that includes: preparing a first report that provides a diagnosis for a disorder based on sequence information about the subject, the sequence information including information about a gene; storing the sequence information about the subject; updating a system that stores information about variants in the gene with data external to said system; determining if a change in the system of variants alters the diagnosis for the disorder as reported for the subject in the first report; and optionally, preparing a subsequent report for the subject that provides a diagnosis for the disorder based on evaluating the subject's sequence information using the updated system. In one embodiment, the data that is used to update the system is acquired from other test subjects and/or from new knowledge from scientific literature or other sources.

In one embodiment, the second or subsequent report is prepared if the system detects an alteration in the level of confidence or an alteration in the database of variants. In another embodiment, the subsequent report is prepared whether or not the level of confidence is altered. For example, the subsequent report includes information that the level of confidence in the diagnosis is unchanged in the case where no alteration is detected. In still other examples, there can be an alteration, but the alteration does not change the level of confidence, although a subsequent report may still be prepared. The table of variants can include references that link a particular variant to stored sequence or clinical information about subjects that have the particular variant. The clinical information or the sequence information about each subject can be stored in the database.

The method can further include requesting and/or receiving information from physician or subject. For example, the request or receipt is made if the subject has a variant that has not been correlated with the disorder at the time of the first report. The method can include other features described herein.

In another aspect, the invention features a server that stores a database comprising records, each record comprising or associating an identifier, genetic information, and phenotypic information, and audit information. For example, the audit information can include date/time information, a checksum, a version number, or a reference associated with a frozen snapshot of a database.

In another aspect, the invention features a system that includes: a database of sequence information that associates identifiers for individuals and sequence information for one or more genes that are associated with a disorder; a database of variants that associates variants in the one or more genes and the disorder, and, e.g., the level of confidence of the association; and one or more processors, configured to access each of the databases and execute a method that includes:
(i) receiving sequence information and clinical information for a subject;
(ii) appending, to the database of sequence information, a record that associates an identifier for the subject and the received sequence information;
(iii) identifying one or more variants in the received sequence information;
(iv) if the identified variant(s) is present in the database, retrieving an indication of the level of confidence that the variant is associated with the disorder from the database of variants and generating a report that comprises the retrieved information; and
(v) determining, from the sequence information and the clinical information for the subject, if the database of variants requires modification. The system can include other features described herein.

In one aspect, the invention features a method for diagnosing and reporting a level of confidence in the diagnosis of a disorder. The method includes: providing a database of variants, the database comprising associations between one or more variants, e.g., in a gene, and the disorder, wherein at least one of the associations comprises a characterization of quality of the associations; determining the sequence of a target region of the gene in a subject, thereby providing sequence information for each subject of multiple subjects; and providing a report for each subject that comprises information about the subject's sequence and the level of confidence in the diagnosis of the disorder as determined by comparing the subject's sequence information to information about associated levels of confidence annotated in the database of variants. The method can include other features described herein.

Another featured method includes: evaluating a study that provides an association between a variant and a disorder to obtain a qualitative or quantitative indicator of quality for the association; modifying a database of variants such that the database stores the association and the indicator of quality; determining the sequence of a target region of the gene in a subject, thereby providing sequence information for multiple subjects; and providing a report for each subject that comprises information about the subject's sequence and the level of confidence in the diagnosis of the disorder as determined by comparing the subject's sequence information to information about associated levels of confidence annotated in the database of variants. In one embodiment, the indicator of quality is based on a linear weighting of a parameter described herein, or two or more parameters described herein. The method can include other features described herein.

In one aspect, the invention features a method that includes: periodically assessing a database or an online-index of biomedical information to identify information about a gene, e.g., information that is new relative to a previous assessment; evaluating the new information using stringency criteria; generating a test rule based on the new information; and processing a database of genetic information in which records for individuals associate genetic information to phenotypic information using the test rule.

In one aspect, the invention features a method that includes: assessing (e.g., periodically) a database or an online-index of biomedical information to identify information about a gene, e.g., information that is new relative to a previous assessment; evaluating the new information using stringency criteria; and producing an alert or other information, e.g., a cost assessment of a diagnostic test. The cost assessment can be based on the new information, e.g., and can also be a function of demographics, reagent costs, accuracy estimation, risk costs, e.g., for failure to diagnose, and so forth. The method can include other features described herein.

In one aspect, the invention features a method of evaluating raw sequencing information. The method includes: comparing the raw sequence information to rules trained with knowledge of the known alleles of the sequence. The method can include other features described herein.

In one aspect, the invention features a method that includes: providing a system that includes a first set of records (gene annotation) and a second set of records (variant database); detecting changes in database; and evaluating correlations between one or more of: gene variants/phenotypes, phenotypes—phenotypes, or gene variants—gene variants.

In one embodiment, the method can include receiving phenotypic information or genetic information, e.g., from a first party, e.g., a client, a doctor, or a patient. The method can include providing a report, e.g., to a party, e.g., a client, a doctor, or a patient. The method can include other features described herein.

The methods described herein can be used for any gene or genes, e.g., any gene or genes associated or suspected of being associated with a disorder. Exemplary disorders include an adrenal disorder (e.g. primary adrenal insufficiency, congenital adrenal hyperplasia ), a lipid disorder (e.g. hypercholesterolemia or dyslipidemia), a bone disorder (e.g. osteoporosis, osteogenesis imperfecta or hypophosphatemic rickets), obesity, a sugar disorder (e.g. hypoglycemia), or other endocrine or metabolic disorder listed in Table 1 or a disorder of the immune system or a disorder of the cardiovascular system. In one embodiment, the lipid disorder is hypercholesterolemia. Exemplary genes associated with hypercholesterolemia include at least one of the following: LDL-R or APOB. In another embodiment, the lipid disorder is dyslipidemia. Exemplary genes associated with dislipidmia include at least one of the following: APA1, ABCA1, LCAT, CETP. In another embodiment, the adrenal disorder is congenital adrenal hyperplasia. Exemplary genes associated with congenital adrenal hyperplasia include at least one of the following: CYP21A2, CYP11B1 or HSD3B2. In other embodiments, the disorder is one of those listed in Table 1 and exemplary genes listed in Table 1 associated with those disorders. The following is a table of exemplary genes and disorders:

TABLE 1

| Gene | Alternate name | Disorder |
|---|---|---|
| FGFR3 | ACH; CEK2; JTK4; | Achondroplasia |
| | HSFGFR3EX | |
| POMC | MSH; POC; ACTH; CLIP | ACTH deficiency |
| TBX19 | TPIT; TBS19; TBS 19; | ACTH deficiency |
| | dJ747L4.1 | |
| CBG | SERPINA6 | adrenal disorder |
| AAAS | AAA; GL003; ADRACALA; | Adrenal Insufficiency |
| | ADRACALIN; | |
| | DKFZp586G1624 | |
| ABCD1 | ALD; AMN; ALDP; ABC42 | Adrenal insufficiency |
| AIRE | APS1; APSI; PGA1; APECED | Adrenal insufficiency |
| MC2R | ACTHR | Adrenal insufficiency |
| NR0B1 | AHC; AHX; DSS; GTD; HHG; | Adrenal insufficiency |
| | AHCH; DAX1 | |
| NR5A1 | ELP; SF1; FTZ1; SF-1; AD4BP; | Adrenal insufficiency |
| | FTZF1 | |
| NR5A1 | ELP; SF1; FTZ1; SF-1; AD4BP; | Adrenal insufficiency |
| | FTZF1 | |
| POMC | MSH; POC; ACTH; CLIP | Adrenal insufficiency |
| STAR | STARD1 | Adrenal Insufficiency |
| TPIT | TBX19; TBS19; TBS 19; | Adrenal Insufficiency |
| | dJ747L4.1 | |
| CRH (4 isoforms) | CRF | Adrenal insufficiency-secondary |
| ACOX1 | ACOX; MGC1198; PALMCOX | ALD |
| PEX1 | ZWS1 | ALD |
| PEX10 | NALD; RNF69; MGC1998 | ALD |
| PEX13 | ZWS; NALD | ALD |
| PXR1 | PEX5, PTS1R | ALD |
| AMH | MIF; MIS | Ambiguous genitalia |
| AMHR2 | AMHR; MISRII | Ambiguous genitalia |
| AR | KD; AIS; TFM; DHTR; SBMA; | Ambiguous genitalia |
| | NR3C4; SMAX1; HUMARA | |
| BBS2 | BBS; MGC20703 | Ambiguous genitalia |
| DMRT1 | DMT1 | Ambiguous genitalia |
| LHCGR | LHR; LCGR; LGR2 | Ambiguous genitalia |
| NR0B1 | AHC; AHX; DSS; GTD; HHG; | Ambiguous genitalia |
| | AHCH; DAX1 | |
| SF1 | ZFM1; ZNF162; D11S636 | Ambiguous genitalia |
| SRA2 | TDFA | Ambiguous genitalia |
| SRD5A2 | | Ambiguous genitalia |
| SRY | TDF, TDY | Ambiguous genitalia |
| SRY | TDF, TDY | Ambiguous genitalia |
| AGL | GDE | Amylo-1,6-glucosidase, 4-alpha- |
| | | glucanotransferase (glycogen |
| | | depranching enzyme) |
| AIRE | APS1; APSI; PGA1; APECED | Autoimmune polyglandular |
| | | syndrome |
| HBB | hemoglobin | Blood disorder |
| ALPL | HOPS; TNAP; TNSALP; AP- | Bone Disorder |
| | TNAP | |
| CALCA | CT; KC; CGRP; CALC1; | Bone Disorder |
| | CGRP1; CGRP-I | |
| COL5A1 | | Bone Disorder |
| FBN1 | FBN; SGS; WMS; MASS; | Bone Disorder |
| | MFS1; OCTD | |
| OPPG | OPS | Bone Disorder |
| PDB | PDB1 | Bone Disorder |
| TNFRSF11A | EOF; FEO; OFE; ODFR; PDB2; | Bone Disorder |
| | RANK; TRANCER | |
| CYP11B1 | FHI; CPN1; CYP11B; P450C11 | CAH |
| CYP17-CYP17A1 | CPT7; CYP17A1; S17AH; | CAH |
| | P450C17 | |
| CYP21A2 | CAH1; CPS1; CA21H; CYP21; | CAH |
| | CYP21B; P450c21B | |
| HSD3B2 | HSDB; HSDB3 | CAH |
| CASR | | Calcium-disorder |
| CASR | FHH; HHC; HHC1; NSHPT; | calcium-disorder |
| | PCAR1; GPRC2A | |
| DGS | DGCR; VCF; CATCH22 | Calcium-disorder |
| DGS2 | DGCR2 | Calcium-disorder |
| GATA3 | HDR; MGC2346; MGC5199; | Calcium-disorder |
| | MGC5445 | |
| GNAS | AHO; GSA; GSP; POH; GPSA; | Calcium-disorder |
| | NESP; GNAS1; PHP1A; PHP1B; | |
| | GNASXL; NESP55 | |
| HCA1 | | Calcium-disorder |
| HHC2 | FBH; FBH2; FHH2 | Calcium-disorder |
| HHC3 | FBH3; FBHOk | Calcium-disorder |
| HRD | | Calcium-disorder |
| HRPT2 | HPT-JT; C1orf28; FLJ23316 | Calcium-disorder |
| PTH | | Calcium-disorder |
| MC1R | MSH-R; MGC14337 | cancer |
| MEN1 | MEAI; SCG2 | cancer |
| MTACR1 | WT2; ADCR | Cancer |
| TP53 | p53; TRP53 | cancer |
| AVP | VP; ADH; ARVP; AVRP; AVP- | Central diabetes insipidus |
| | NPII | |
| ACG1A | | Collagen |
| ADAMTS2 | NPI; PCINP; PCPNI; hPCPNI; | Collagen |
| | ADAM-TS2; ADAMTS-3 | |
| COL2A1 (2 | SEDC; COL11A3 | Collagen |
| isoforms) | | |
| COL3A1 | EDS4A | Collagen |
| COL5A2 | | Collagen |
| PLOD | LH; LLH; PLOD1 | Collagen |
| SLC26A2 | DTD; EDM4; DTDST; MST153; | Collagen |
| | D5S1708; MSTP157 | |
| LHX3 | M2-LHX3 | Combined Pituitary Hormone |
| | | Deficiency |
| POU1F1 | PIT1; GHF-1 | Combined Pituitary Hormone |
| | | Deficiency |
| POU1F1 | PIT1; GHF-1 | Combined Pituitary Hormone |
| | | Deficiency |
| PROP1 | None | Combined Pituitary Hormone |
| | | Deficiency |
| PROP1 | | Combined Pituitary Hormone |
| | | Deficiency |
| DUOX2 | LNOX2; THOX2; NOXEF2; | Congenital hypothyroidism |
| | P138-TOX | |
| PAX8 | | Congenital hypothyroidism |
| TG | AITD3 | Congenital hypothyroidism |
| TPO | MSA; TPX | Congenital hypothyroidism |
| TSHR | LGR3 | Congenital hypothyroidism |
| CNC2 | | Cushing syndrome |
| GNAI2 | GIP; GNAI2B | Cushing syndrome |
| PRKAR1A | CAR; CNC1; PKR1; TSE1; | Cushing's syndrome |
| | PRKAR1; MGC17251 | |
| AIR | | Diabetes Mellitus |
| CAPN10 | | Diabetes mellitus |
| IB1 | MAPK8IP1; JIP-1; PRKM8IP | Diabetes mellitus |
| IDDM10 | | Diabetes mellitus |
| IDDM11 | | Diabetes mellitus |
| IDDM12 | | Diabetes mellitus |
| IDDM13 | | Diabetes mellitus |
| IDDM15 | | Diabetes mellitus |
| IDDM17 | | Diabetes mellitus |
| IDDM18 | | Diabetes mellitus |
| IDDM2 | IDDM; ILPR; IDDM1 | Diabetes mellitus |
| IDDM3 | | Diabetes mellitus |
| IDDM4 | | Diabetes mellitus |
| IDDM5 | | Diabetes mellitus |
| IDDM6 | | Diabetes mellitus |
| IDDM7 | | Diabetes mellitus |
| IDDM8 | | Diabetes mellitus |
| IDDMX | | Diabetes mellitus |
| INSR | | Diabetes mellitus |
| IRS1 | HIRS-1 | Diabetes mellitus |
| PPARG | NR1C3; PPARG1; PPARG2; | Diabetes mellitus |
| | HUMPPARG | |
| DHS | DHS | Electrolyte disorder |
| CACNA1S | MHS5; HOKPP; hypoPP; | Electroyle-disorder |
| | CCHL1A3; CACNL1A3 | |
| CLDN16 | PCLN1 | Electroyle-disorder |
| FXYD2 | HOMG2; ATP1G1; MGC12372 | Electroyle-disorder |
| HOMG | TRPM6; HSH; HMGX; CHAK2; | Electroyle-disorder |
| | FLJ20087; FLJ22628 | |
| KCNE3, HOKPP | MIRP2 | Electroyle-disorder |
| SCN4A | HYPP; HYKPP; NAC1A; | Electroyle-disorder |
| | Nav1.4; hNa(V)1.4 | |
| MENIN | MEA1, ZES, MEN1 - Not listed | Endocrine cancer |
| | in “Gene” database | |
| RET | PTC; MTC1; HSCR1; MEN2A; | Endocrine cancer |
| | MEN2B; RET51; CDHF12 | |
| SDHD | PGL; CBT1; PGL1; SDH4 | Endocrine cancer |
| NTRK1 | MTC; TRK; TRKA | endocrine-cancer |
| AR | KD; AIS; TFM; DHTR; SBMA; | Endocrine-cancer: |
| | NR3C4; SMAX1; HUMARA | |
| GHRH | GRF; GHRF | Growth |
| GRB10 | RSS; IRBP; MEG1; GRB-IR; | Growth |
| | KIAA0207 | |
| PTPN11 | CFC; NS1; SHP2; BPTP3; | Growth |
| | PTP2C; PTP-1D; PRO1847; SH- | |
| | PTP2; SH-PTP3; MGC14433 | |
| SMTPHN | | Growth, Tall Stature, Endocrine |
| | | Tumor |
| G6PC | G6PT; GSD1a | Glycogen Storage Disease |
| G6PT/G6PT1 | G6PC | Glycogen Storage Disease |
| G6PT1 | | Glycogen Storage Disease |
| GAA | LYAG | Glycogen Storage Disease |
| GBA | GCB; GBA1; GLUC | Glycogen Storage Disease |
| GBE1 | GBE | Glycogen Storage Disease |
| GYS2 | | Glycogen Storage Disease |
| LAMP2 | LAMPB; CD107b | Glycogen Storage Disease |
| PFKM | MGC8699 | Glycogen Storage Disease |
| PHKA2 | PHK; PYK; XLG; PYKL; XLG2 | Glycogen Storage Disease |
| PHKG2 | | Glycogen Storage Disease |
| CYP11B1 | FHI; CPN1; CYP11B; P450C11 | Hirsuitism |
| CYP21A2 | CAH1; CPS1; CA21H; CYP21; | Hirsuitism |
| | CYP21B; P450c21B | |
| HSD3B2 | HSDB; HSDB3 | Hirsutism |
| NR3C1 | GR; GCR; GRL | Hirsutism |
| ELN | WS; WBS; SVAS | Hypercalcemia |
| AGTR1 | AT1; AG2S; AT1B; AT2R1; | Hypertension |
| | HAT1R; AGTR1A; AGTR1B; | |
| | AT2R1A; AT2R1B | |
| BSND | BART | Hypertension |
| CLCNKB | CLCKB; hClC-Kb | Hypertension |
| COL3A1 | EDS4A | Hypertension |
| CYP11B1.B2 fusion | | Hypertension |
| CYP11B2 | CPN2; ALDOS; CYP11B; | Hypertension |
| | CYP11BL; P-450C18; P450aldo | |
| CYP17-CYP17A1 | CPT7; CYP17A1; S17AH; | Hypertension |
| | P450C17 | |
| FHII | FHA2 | Hypertension |
| HTNB | | Hypertension |
| HYT1 | | Hypertension |
| HYT2 | | Hypertension |
| NPR3 | NPRC; ANPRC | Hypertension |
| PEE1 | PEE, PREG1 | Hypertension |
| PHA2 | PHA2A | Hypertension |
| PHA2C | PRKWNK1; KDP; WNK1; | Hypertension |
| | KIAA0344 | |
| PNMT | PENT | Hypertension |
| PRKWNK4 | WNK4; PHA2B | Hypertension |
| SCNN1A | ENaCa; SCNEA; SCNN1; | Hypertension |
| | ENaCalpha | |
| SCNN1B | ENaCb; SCNEB; ENaCbeta | Hypertension |
| SCNN1B | ENaCb; SCNEB; ENaCbeta | Hypertension |
| SCNN1G | PHA1; ENaCg; SCNEG; | Hypertension |
| | ENaCgamma | |
| SCNN1G | PHA1; ENaCg; SCNEG; | Hypertension |
| | ENaCgamma | |
| SLC12A3 | TSC; NCCT | Hypertension |
| CYP11B1 | FHI; CPN1; CYP11B; P450C11 | Hypertension |
| HSD11B2 | AME; AME1; HSD11K | Hypertension |
| NR3C1 | GR; GCR; GRL | Hypertension |
| ABCC8 | HI; SUR; MRP8; PHHI; SUR1; | Hypoglycemia |
| | ABC36; HRINS | |
| GCK | GK; GLK; HK4; HKIV; HXKP; | Hypoglycemia |
| | MODY2; NIDDM | |
| GLUD1 | GDH; GLUD | Hypoglycemia |
| KCNJ11 | BIR; PHHI; IKATP; KIR6.2 | Hypoglycemia |
| PCK1 | PEPCK1, PEPKC, PEPCK | Hypoglycemia |
| SLC22A5 | OCTN2 | Hypoglycemia |
| CYP19 | ARO; ARO1; CPV1; CYAR; | Hypogonadism |
| | CYP19A1; P-450AROM | |
| GNRHR | GRHR; LHRHR | Hypogonadism |
| KAL1 | KMS, KALIG1, ADMLX | Hypogonadism |
| LHCGR | LHR; LCGR; LGR2 | Hypogonadism |
| NR0B1 | AHC; AHX; DSS; GTD; HHG; | Hypogonadism |
| | AHCH; DAX1 | |
| NR5A1 | ELP; SF1; FTZ1; SF-1; AD4BP; | Hypogonadism |
| | FTZF1 | |
| STAR | STARD1 | Hypogonadism |
| FGF23 | ADHR; HYPF; HPDR2 | Hypophasphatemic Rickets |
| PHEX | HYP; PEX; XLH; HPDR; HYP1; | Hypophosphatemic rickets |
| | HPDR1 | |
| INSR | None | Insulin resistance |
| ABCA1 | TGD; ABC1; CERP; HDLDT1 | Lipid |
| APOA1 | | Lipid |
| APOA2 | | Lipid |
| APOB | FLDB | Lipid |
| APOC3 | | Lipid |
| CETP | | Lipid |
| FH3 | PCSK9; NARC1; HCHOLA3 | Lipid |
| FHCB1 | ARH1 | Lipid |
| HADHA | GBP; MTPA; LCHAD | Lipid |
| HYPLIP1 | USF1; UEF; MLTF; FCHL1; | Lipid |
| | MLTFI | |
| HYPLIP2 | FCHL2 | Lipid |
| LCAT | | Lipid |
| LDLR | FH; FHC | Lipid |
| LPL | LIPD | Lipid |
| UGT1A1 | GNT1; UGT1; UDPGT; UGT1A; | Liver disorder |
| | UGT1*1; HUG-BR1 | |
| CFTR | CF; MRP7; ABC35; ABCC7 | Male infertility |
| PAH | PKU; PKU1 | Metabolic disorder |
| GCK (3 isoforms) | GK; GLK; HK4; HKIV; HXKP; | MODY |
| | MODY2; NIDDM | |
| HNF4A | TCF; HNF4; NR2A1; TCF14; | MODY |
| | HNF4a9; NR2A21 | |
| INS | | MODY |
| IPF1 | IUF1; PDX1; IDX-1; MODY4; | MODY |
| | PDX-1; STF-1 | |
| TCF1 | HNF1; LFB1; HNF1A; MODY3 | MODY |
| TCF2 | HNF2; LFB3; HNF1B; MODY5; | MODY |
| | VHNF1; HNF1beta | |
| ADL/SGCA | A2; ADL; DAG2; DMDA2; 50- | Muscle disorder |
| | DAG; LGMD2D; SCARMD1; | |
| | adhalin | |
| GCK (3 isoforms) | GK; GLK; HK4; HKIV; HXKP; | Neonatal diabetes |
| | MODY2; NIDDM | |
| IPF1 | IUF1; PDX1; IDX-1; MODY4; | Neonatal diabetes |
| | PDX-1; STF-1 | |
| AQP2 | AQP-CD; WCH-CD; MGC34501 | Nephrogenic diabetes insipidus |
| AVPR2 | DI1; DIR; NDI; V2R; ADHR; | Nephrogenic diabetes insipidus |
| | DIR3 | |
| SLS/ALDH3A2 | FALDH; ALDH10 | Neuro disorder |
| AQP1 | CO; CHIP28; AQP-CHIP; | Normal |
| | MGC26324 | |
| REN | | Normal |
| ADRB2 | BAR; B2AR; ADRBR; | Obesity |
| | ADRB2R; BETA2AR | |
| BBS1 | BBS2L2; FLJ23590 | Bardet-Biedl Syndrome |
| BBS2 | BBS; MGC20703 | Bardet-Biedl Syndrome |
| BBS3 | ARL6, MGC32934 | Bardet-Biedl Syndrome |
| BBS4 | None | Bardet-Biedl Syndrome |
| BBS5 | DKFZp762I194 | Bardet-Biedl Syndrome |
| BBS6 | MKKS, KMS; MKS; BBS6; | Bardet-Biedl Syndrome |
| | HMCS | |
| CDKN1C | BWS; WBS; p57; BWCR; KIP2 | obesity |
| CRBM | SH3BP2; CRPM; RES4-23 | Obesity |
| GNAS | AHO; GSA; GSP; POH; GPSA; | Obesity |
| | NESP; GNAS1; PHP1A; PHP1B; | |
| | GNASXL; NESP55 | |
| GNB3 | | Obesity |
| LEP | OB; OBS | Obesity |
| MC4R | | Obesity |
| MKKS | KMS; MKS; BBS6; HMCS | Bardet-Biedl Syndrome |
| NR0B2 | SHP; SHP1 | Obesity |
| OB10 | OB10P | Obesity |
| OQTL | OB20 | Obesity |
| PCSK1 | PC1; PC3; NEC1; SPC3 | Obesity |
| POMC | MSH; POC; ACTH; CLIP | Obesity |
| PPARG | NR1C3; PPARG1; PPARG2; | Obesity |
| | HUMPPARG | |
| SIM1 | | Obesity |
| NDN | HsT16328 | Obesity, Reproductive |
| PWS | PWCR | Obesity, Reproductive |
| SNRPN | SMN; SM-D; HCERN3; | Obesity, Reproductive |
| | SNRNP-N; SNURF-SNRPN | |
| COL1A1 | OI4 | Osteogenesis Imperfecta |
| COL1A2 | OI4 | Osteogenesis Imperfecta |
| COL1A1 | OI4 | Osteoporosis |
| LRP5 | HBM; LR3; OPS; LRP7; OPPG; | Osteoporosis |
| | BMND1; VBCH2 | |
| FOXC1 | ARA; IGDA; IHG1; FKHL7; | Pituitary-disorder |
| | IRID1; FREAC3 | |
| PITX2 | RS; RGS; ARP1; Brx1; IDG2; | Pituitary-disorder |
| | IGDS; IHG2; PTX2; RIEG; | |
| | IGDS2; IRID2; Otlx2; RIEG1; | |
| | MGC20144 | |
| PRKCA | PKCA; PRKACA; PKC-alpha | Pituitary-disorder |
| RIEG2 | ARS; RGS2 | Pituitary-disorder |
| CYP11B1 | FHI; CPN1; CYP11B; P450C11 | Precocious puberty (boys) |
| CYP21A2 | CAH1; CPS1; CA21H; CYP21; | Precocious puberty (boys) |
| | CYP21B; P450c21B | |
| LHCGR | LHR; LCGR; LGR2 | Precocious puberty (boys) |
| HSD3B2 | HSDB; HSDB3 | Precocious puberty (males) |
| NR3C1 | GR; GCR; GRL | Precocious Puberty (males) |
| AGT | ANHU; SERPINA8 | pregnancy disorder |
| CSH1 | PL; CSA; CSMT | pregnancy disorder |
| NOS3 | eNOS; ECNOS | pregnancy disorder |
| HSD3B2 | HSDB; HSDB3 | Premature Adrenarch (both |
| | | genders) |
| CYP11B1 | FHI; CPN1; CYP11B; P450C11 | Premature adrenarche |
| CYP21A2 | CAH1; CPS1; CA21H; CYP21; | Premature adrenarche |
| | CYP21B; P450c21B | |
| NR3C1 | GR; GCR; GRL | Premature adrenarche |
| ESR1 | ER; ESR; Era; ESRA; NR3A1 | Reproductive |
| GALT | | Reproductive |
| CYP11A1 | CYP11A; P450SCC | Reproductive - F |
| DIAPH2 | DIA; POF; DIA2; POF2 | Reproductive - F |
| FSHR | LGR1; ODG1; FSHRO | Reproductive - F |
| FST (2 isoforms) | FS | Reproductive - F |
| ACR | | Reproductive - M |
| AZF1 | AZF; SP3; AZFA | Reproductive - M |
| FSHB | | Reproductive - M |
| HSD17B3 | EDH17B3 | Reproductive - M |
| LHB | CGB4; LSH-B | Reproductive - M |
| UBE2B | HR6B; UBC2; HHR6B; RAD6B; | Reproductive - M |
| | E2-17 kDa | |
| DAZ | DAZ1; SPGY | Reproductive - M; Male |
| | | infertility with azoospermia |
| AR | KD; AIS; TFM; DHTR; SBMA; | Reproductive, ambiguous |
| | NR3C4; SMAX1; HUMARA | genitalia |
| DHH | HHG-3; MGC35145 | Reproductive, ambiguous |
| | | genitalia |
| GDXY | GDXY; SRVX; TDFX | Reproductive, ambiguous |
| | | genitalia |
| CYP27B1 | VDR; CP2B; CYP1; PDDR; | Rickets |
| | VDD1; VDDR; VDDRI; | |
| | CYP27B; P450c1; VDDR I | |
| VDR | NR1I1 | Rickets |
| CYP11B2 | CPN2; ALDOS; CYP11B; | Salt losing syndrome of the |
| | CYP11BL; P-450C18; P450aldo | newborn |
| NR3C2 | MR; MCR; MLR | Salt losing syndrome of the |
| | | newborn |
| GH1 (5 isoforms) | GH; GHN; GH-N; hGH-N | Short stature |
| GHR | | Short stature |
| GHRHR | GHRFR | Short stature |
| GNAS | AHO; GSA; GSP; POH; GPSA; | Short stature |
| | NESP; GNAS1; PHP1A; PHP1B; | |
| | GNASXL; NESP55 | |
| IGF1 | IGFI | Short stature |
| SHOX | SS; GCFX; PHOG; SHOXY | Short Stature |
| SLC2A1 | GLUT; GLUT1 | Sjogren-Larsson Syndrome |
| NSD1 | STO; SOTOS; ARA267; | Sotos syndrome |
| | FLJ22263 | |
| GRD2 | | Thyroid |
| MNG1 | | Thyroid |
| MNG2 | | Thyroid |
| ALB | PRO0883 | Thyroid binding abnormalities |
| TBG | SERPINA7 | Thyroid binding abnormalities |
| TTR | PALB; TBPA; HsT2651 | Thyroid binding abnormalities |
| THRB | GRTH; THR1; ERBA2; NR1A2; | Thyroid hormone resistance |
| | THRB1; THRB2; ERBA-BETA | |
| D10S170 | CCDC6; H4; PTC; TPC; TST1; | Thyroid Hypothryoid |
| | D10S170 | |
| SLC5A5 | NIS | Thyroid Hypothryoid |
| TSHB | TSH-BETA | Thyroid Hypothryoid |
| PTCPRN | PRN1 | Thyroid Hypothryoid; Abnormal |
| | | TFT's |
| SERPINA7 | TBG | Thyroid Hypothryoid; Abnormal |
| | | TFT's |
| TITF1 | BCH; BHC; NK-2; TEBP; TTF1; | Thyroid -hypothyroid |
| | NKX2A; TTF-1; NKX2.1 | |
| TRH | | Thyroid -hypothyroid |
| TCO | TCO1 | Thyroid, endocrine cancer |
| TSHR | LGR3 | Thyroid, endocrine cancer |
| CYP17-CYP17A1 | CPT7; CYP17A1; S17AH; | Undervirilized male/ambiguous |
| | P450C17 | genitalia |
| HSD3B2 | HSDB; HSDB3 | Undervirilized male/ambiguous |
| | | genitalia |
| STAR | STARD1 | Undervirilized male/ambiguous |
| | | genitalia |
| WFS1 | WFS; WFRS; DFNA6; DFNA14; | Wolfram syndrome |
| | DFNA38; DIDMOAD; | |
| | WOLFRAMIN | |
| CYP2C9 | CPC9; CYP2C10; P450IIC9; | |
| | P450 MP-4; P450 PB-1 | |
| HCRT | OX; PPOX | |
| HEXA | TSD | |
| NPC1 | NPC | |
| TTF1 | BCH; BHC; NK-2; TEBP; TTF1; | |
| | NKX2A; TTF-1; NKX2.1 | |

This application incorporates all patents, applications, and references mentioned herein, including U.S. Application Serial No. 60/529,274, filed on 12 Dec. 2003, Ser. No. 60/550,784, filed Mar. 5, 2004, Ser. No. 60/591,668, filed on 28 Jul. 2004, and Ser. No. ______, filed Dec. 10, 2004, bearing attorney docket number 13154-013001, titled “Sequencing Data Analysis.”

### BRIEF DESCRIPTION OF THE DRAWINGS

FIG. 1 depicts a schematic of a first exemplary system for processing and managing genetic information.

FIG. 2 depicts a schematic of a database for managing genetic information.

FIG. 3 depicts a schematic of a second exemplary systems for processing and managing genetic information.

### EXAMPLE I

The method and systems described herein can be implemented in a variety of ways. This disclosure includes two non-limiting examples that illustrate particular implementations that can be used. Other implementations can include one or more features that are described herein.

These implementation can be used, inter alia, to automatically revise interpretation of the patient's sequence based on revisions in correlation coefficients of a curated database of variants, for example, to make an initial diagnosis and then to repeatedly revise the diagnosis or degree of confidence in a diagnosis using patient's gene sequence information obtained in connection with the initial testing and a database of variants that changes over time. Since a patient's gene sequence typically does not change with time, sequence information can be stored and used at later times, e.g., in combination with new information.

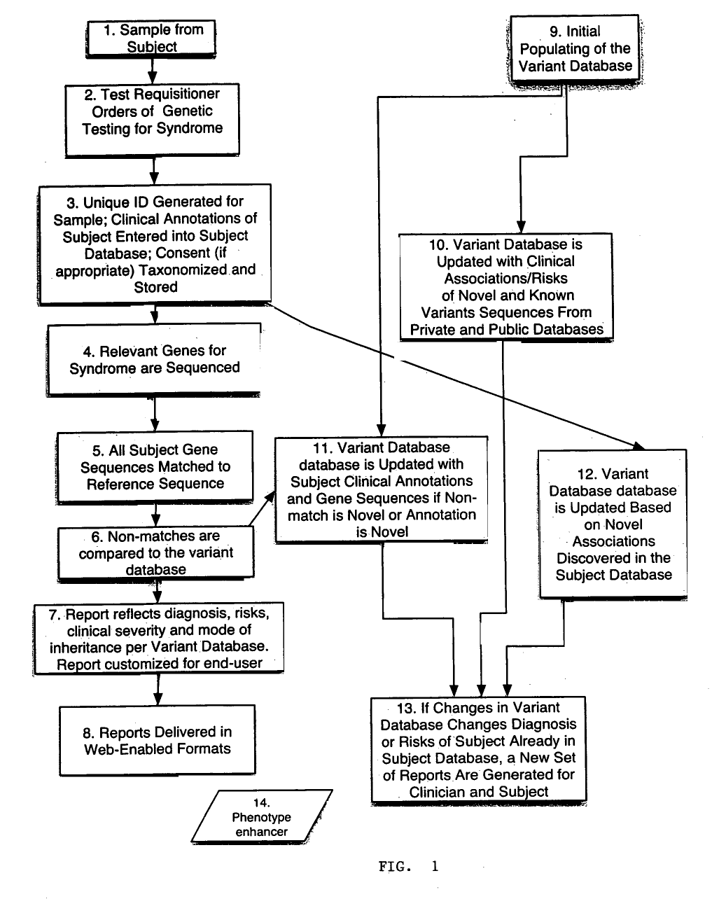

One exemplary implementation, described in FIG. 1, includes the following processes:

Process 1. A sample is obtained from the subject. The subject is also evaluated to obtain information about phenotype, for example, historical items, family history, physical exam, biochemical studies, expression studies, proteomic studies. The phenotypic information can be obtained as deemed relevant per protocol for the disorder in question.

Process 2: A test requisitioner (e.g., researcher, research assistant, clinician or automated computer console, or web page) can obtain:

Consent (if necessary) with a formalized description of what additional uses can be made of the samples and phenotypic annotations and under what conditions, if any, the subject, directly or through clinician, can, should or will be informed regarding novel findings related to their genetic status and whether or not they may be approached for additional phenotypic data.

The subject phenotypic data is in a standardized format and mapped into the appropriate standardized nomenclature. The data is entered into an electronic order system or a paper-based order system. If paper-based, an assistant will enter the data into the electronic system or the paper can be electronically scanned or captured. If there are any missing data or additional data required, the test requisitioner is prompted for these prior to the end of the initial ordering transaction. The minimal phenotypic annotation sample can be determined as the union of a core data set required of all orders and a templated additional data set that is specific to the disorder for which testing has been ordered.

Process 3: Entry of subject data and order into the Subject Database. A Unique ID for each subject is generated. Associated with this ID are all the phenotypic data, the accession numbers and sample information for the subject sample.

Process 4: For all genes requisitioned to be associated with the disorder for which the subject is to be tested, each gene is sequenced. The sequencing includes any part or all of the coding regions of the gene and any part or all of the identified regulatory regions (in introns or promoter regions or 3′ untranslated region) reference sequences are defined with respect to the NIH's reference sequence database. The raw data from sequencing is stored in the Subject Database as are the bases “called” for the Subject's DNA sequence. The base calling procedure is informed by the known reference sequence in the Variant Database (See Process 9, below) such that ambiguous base calls can be disambiguated based on the prior knowledge constituted by the reference sequence. The called bases are stored in the Subject Database. We refer to the string of bases called for a particular gene the “base called sequence.”

Process 5: The base called sequence from Process 4 is compared using exact string matching against the reference sequence for each corresponding gene (as annotated in the Variant Database as described in Process 9). The start and end location of each change is noted by nucleotide position on the reference sequence. The changes (substitution, insertion, deletion of bases) at the specified position are also noted in the same standardized genomic nomenclature as is used to populated the Variant Database.

Process 6. If Process 5 notes a deviation of the base called sequence (of the Subject) from the reference sequence, then a lookup function is used to see if any of the variants, noted in Process 5 by standardized variant nomenclature, correspond to a variant specified by standardized variant nomenclature in the Variant Database for the same phenotype as is noted in the Subject Database for that Subject. The standardized variant name is one of the database keys in the Variant Database. All matches of variants in the Variant Database to the base called sequence are noted and a pointer to the relevant annotation data (see Process 9) is maintained for each matching variant.

Process 7: Reporting on variants. The rule-based reporting software assembles fragments of predefined text for each of the levels of certainty, severity, mode of inheritance and other annotations available (see Process 9) for each gene into a coherent formatted report. The rules are developed to be driven by the formally scored annotations in the Variant Database. Several versions of this assembly process can be executed, one for each of the intended readers: clinician, patient/Subject, and researcher etc. The report is reviewed in the context of the electronically reproduced raw sequencing data, the existing annotations, and whatever additional patient data is available. The report is then forwarded to the intended reader. The entire report can be time-stamped electronically authenticated and entered into the patient database.

Process 8: As per end-user preferences and within regulatory framework, reports are delivered in a pre-defined order (e.g. test-requisitioner only, or test-requisitioner followed by Subject) by paper or electronic means. Both media provide guidelines for obtaining more specific information, reminders of the conditions (if any) under which the end-users may or will be recontacted, and availability of various genetic counseling services, if appropriate.

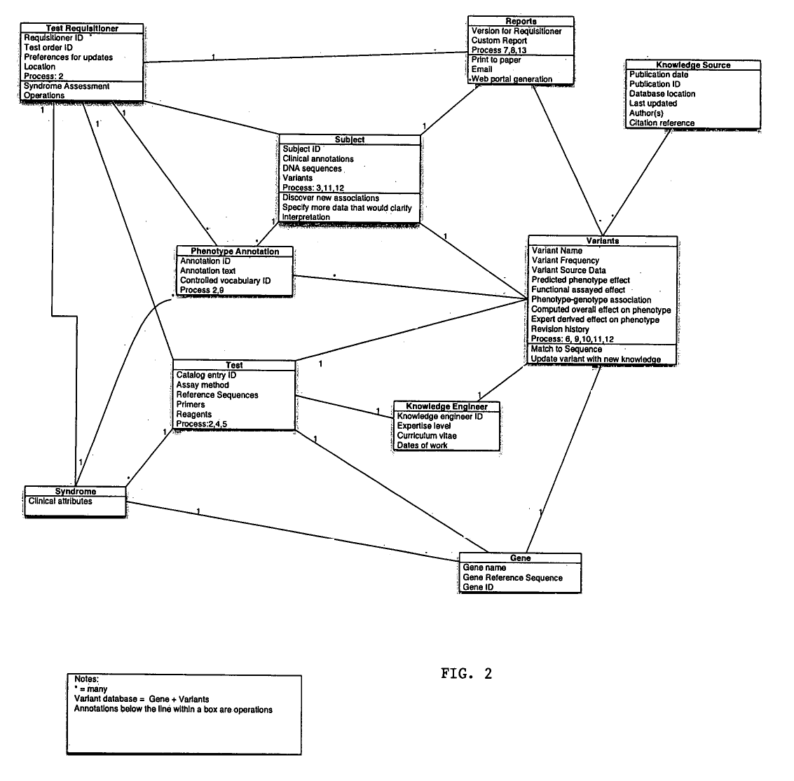

Process 9: Initial populating of the variant database. This database provides knowledge of the clinical consequences (e.g., disease manifestations, physical characteristics, behavior patterns, changes in analytes such as small molecule biochemicals, proteins, RNA expression, etc.) of a variant in DNA sequence. The database can include information about the level of confidence in an association between a variant and a disorder. This database can be initially populated, e.g., using information from the literature. For example, information can be collated by semi-automated procedures (e.g. alerting by software robots of changes in the published literature relevant to a specified gene or variant) and by automated extraction of variant annotations from public and private formally codified databases, and also by manual review. These various information collection processes are used to populate the database to specifications described below. See also, for example, FIG. 2.

This database can contain a reference sequence for each gene (e.g., the coding regions and/or non-coding regions, e.g., regulatory regions).

This database can contain a specification of the exact syntactic nature of the variant using standardized nomenclature for sequence substitution, deletion or insertion. The annotation software ensures that no annotation can be entered that is syntactically invalid or describes sequence that does not correspond to the reference sequence.

The database is populated by classifying each variant using one or more of the following parameters: (1) a parameter indicating the quality of phenotypic-genotypic association based on the knowledge of the pedigree and/or association studies used to populate the database, or an estimate thereof; (2) a parameter indicating the quality of functional studies (e.g. transfection studies, biochemical assays etc.) performed by one or more researchers to determine the functional significance of a particular variant, or an estimate thereof; and (3) a parameter indicating the likelihood that a given variant will cause a change in function and/or phenotype based on the nature of the change of the coded amino acid, the change of a conserved sequence, the chance of an important part of a functional domain of a gene/protein, or an estimate thereof.

For example, the parameter can decrease the level of reliance on an association, e.g., if the study in question was done on small number of subjects or a highly selected population of subjects, e.g., a highly stratified population. The parameter can increase the level of confidence in the diagnosis, if for example it was done on a larger number of subjects, it was performed using a highly relevant population, or if additional studies have corroborated the findings. The parameter can be based on comparisons by those skilled in the art.

This classification is a summary statistic of the aforementioned estimates and allows for a specification of the level of confidence in the diagnosis of the disorder, based on a linear weighting of such estimates.

This output of the database allows for the automatic generation of report that contains one or more of: (i) an indication of the overall importance of the specified variant in causing a specified phenotypic change; and/or (ii) a description of the phenotypic characteristics entailed by each variant using a controlled vocabulary.

This database can contain a list of relevant references for each of the specified variants.

It can include information about (e.g., a quantification of) the number of individuals of families for which such a variant has been reported or found through actual genetic testing. If the variant is not rare an estimate of the percentage of individuals in a specified population is provided.

Process 10: The variant database is maintained to be current so that is contains publicly available variants and annotations as to their phenotypic implications and may also contain variants in private databases and their annotations, to the extent access is obtained. The knowledge engineer responsible for the annotations for a specific gene is notified by software robots that periodically search electronically available sources, e.g., PUBMED®. Any PUBMED® listed publication that includes mention of the gene and variants, polymorphisms, inserts, deletions, and/or mutations in that gene are brought to the attention of the knowledge engineer by means of a software robot using standard text retrieval techniques. For structured data or parse-able text, the information is extracted automatically and as far as is possible transformed into the standardized format of the variant table, e.g., through iterative application of regular expression transformations.

Process 11: The process of matching variants from subject's sample to the Variant Database may fail, if the variant is novel, or the clinical annotation is novel, or both. In these three cases, the non-matching called base sequence with all phenotypic annotations can be presented electronically to the domain expert responsible for that gene or to a module, e.g., that re-evaluates the data or executes a decision. The domain expert or module can decide to either assert that the match already existed but was missed by the matching software (e.g. the phenotype is syntactically but not semantically distinct from prior annotations) or is a novel one. In the latter case, the Variant Database is updated but instead of citing a paper, the subject's record in the Subject Database is referenced.

Process 12: When the Subject Database is updated, all gene variants for all subjects in the Subject Database can be or are re-evaluated. This process detects new or altered statistically significant associations between one or more variants and one or more phenotypic variants. This procedure can be performed using one or both of the Bayesian and frequentist models. For the Bayesian approach, all models/dependencies are evaluated and those dependencies that exceed those of competing models by a defined Bayes factor threshold are selected and submitted to the knowledge engineer for consideration for updating the Variant Database. In the frequentist approach several parametric and non-parametric statistics are applied to determine if, after correction for multiple hypothesis testing, any association exceeds a significance threshold. Application of each of these approaches, in some cases, may not constitute a determination of automatic insertion into the Variant Table but nevertheless provides an indication of an altered, e.g., higher likelihood association from the Subject Database.

Process 13: Updates to the End-User. If Processes 10 and/or 11 cause a change in the Variant Database then the Subject Database is automatically queried to find those Subject's whose Variants match the changed Variant annotation in the Variant Database. The Subject Database is then further queried to determine which of several End-Users can or should be contacted with the updated information (e.g. Test-Requisitioner, Subject, Researcher). New reports (similar to those generated in Process 7 but with highlighting of the new information) can be reviewed and forwarded to the designated End-Users.

### EXAMPLE II

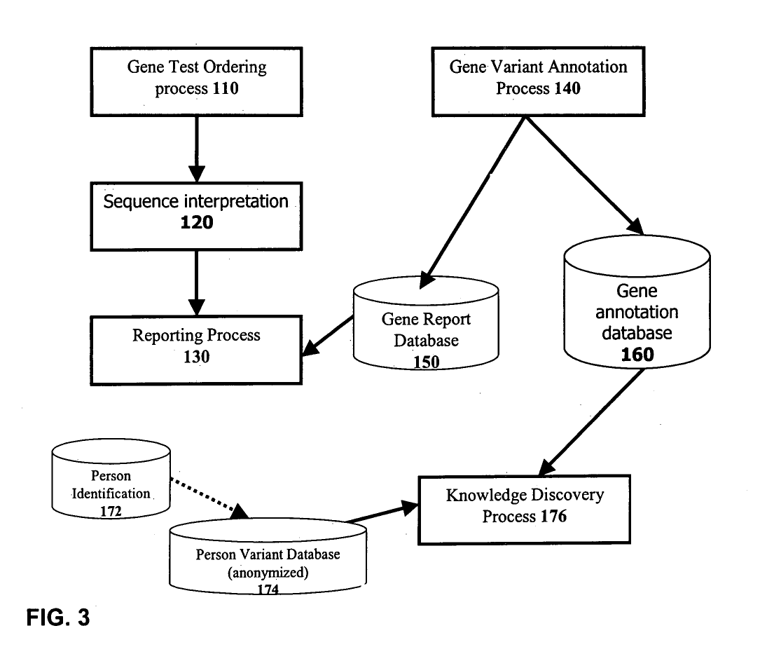

Another implementation, depicted in FIG. 3, is exemplified by “CORD™.” Other embodiments can include one or more features of CORD™.

CORD™ enables a company or laboratory to conduct high quality and high throughput genetic testing. CORD™ can also enable the computational discovery of novel high-yield hypotheses, e.g., for the relationship between specific genotypic data obtained from genetic testing and phenotypic data/disease states, and for genetic modifiers of already known relationships, between specific genotypes and phenotypes. These discoveries can than be used, e.g., to identify pharmacological targets. CORD™ can provide a service that includes comprehensive electronic updating of previous interpretations with then-current knowledge of genotypic-phenotypic associations. This updating service can be used in connection with the diagnosis and treatment planning, and/or genetic counseling of persons that have been tested.

Gene Variant Annotation Process

CORD™ annotates each gene variant to associate the variant with phenotypes. Each phenotype in the database can be associated with one or more gene variant(s). The annotations describe the phenotypic change (e.g. disease) so that there is an authoritative and timely interpretation of all gene variants that may be found through sequencing of DNA. The annotations can include date, checksum, verification, or other audit information

The sources of these annotations can be the CORD™ Biomedical Database Polling and Snapshot software, the CORD™ Knowledge Discovery Process ( see, e.g., below), and the Cord Structured Literature Review Process.

The CORD™ Biomedical Database Polling and Snapshot (BDPS) software has a default but modifiable set of remote third party public and commercial/private databases regarding biomedical research and gene variants in particular that it accesses, e.g., on a regular periodic schedule (the polling cycle). On each of these periodic searches, all information from those databases for all variants of the specified set of genes is retrieved. This constitutes the gene “snapshot” for this polling cycle. A systematic comparison is then done of the retrieved data from each of those databases and the data obtained from the same databases on the prior polling cycle. Any differences found between the snapshots of the two cycles can generate an alert. For example, a difference can be highlighted and a user can be notified. In another embodiment, a difference can trigger an automated process of updating.

The CORD™ Structure Literature Review Process (SLRP) is a multilevel checklist developed to ensure that knowledge workers will obtain all necessary information (or verify its absence) regarding the variants of a gene to permit the user of CORD to provide accurate, complete and timely clinical interpretations of each gene variant specified. It includes questions the knowledge worker must answer in reviewing the literature (which constitutes a subset of the snapshot generated by the BDPS software) for the gene to which they are assigned. The SLRP can include one or more of: the normal physiology of the gene and the patho-physiology of its variants, the differential diagnosis for the pathophysiology, and where applicable, how the test of the genetic variant can be used to improve current diagnostic protocol, e.g., in terms of costs and health benefits.

In one embodiment, a user reviews one or more sources of information on variants of the gene for which she is responsible (e.g., BDPS and SLRP) and updates the CORD™ Gene Annotation Database 160. This database contains, e.g., for each variant of a gene, one or more of: definition of the variant in standard nomenclature; description of all the phenotypic/disease associations known for that variant; quantitative assessment of the incidence of the variant; qualitative assessment of the quality of the evidence for the described association; qualitative assessment of penetrance of the effect of the variant upon the phenotype; qualitative assessment of the importance of the variant in making the diagnosis of the phenotype with which it is associated; and association with one or more pharmacological or therapeutic methods or agents.

In another embodiment, an agent or other computer-based module performs an automated review. For example, the agent can look for new database entries and scan them for useful content. Certain agents can be trained, e.g., using a neural network, genetic algorithm, or other process.

The Gene Report Database 150 is an accessory database for the Gene Annotation Database 160. It contains all the report text templates for each variant. There may be several report types for each gene variant to allow for different report content targeted for different purposes.

Every time the Gene Annotation Database 160 is changed, it is possible to generate an alert. For example, the alert can be directed to an agent (e.g., a computer module or “knowledge worker” or other user). The agent can evaluate if the change in annotation would result in a change of the clinical interpretation of the gene variant. If the agent decides that there is a change in clinical interpretation, the agent can trigger a process whereby one or more (e.g., all) persons who previously received an interpretation on this variant then receive the new information.

Sequence Interpretation Process

Once the specimen is sequenced, the CORD™ Base-Calling Software (BCS) takes as input the trace data in standard format (e.g. from SCF files and ABI model 373 and 377 DNA sequencer chromat files) and interprets 120 the traces to generate a standard sequence file (e.g. in FASTA format). This interpretation is based on the prior probabilities of all the known sequences of gene's variants. That is, the probability of each trace peak corresponding to a particular base is informed by the current base expected in the sequence and the ones identified prior to the current base. This reduces the false positive rate of base calling (and therefore increases the efficiency of the sequence interpretation and validation process 120). Traces which are consistent with deviations from the expected base (e.g., a sequence that has never been seen before throughout the available databases and literature, as documented by the CORD™ gene variant annotation process 140 in the CORD™ Gene Annotation Database 160) generate alerts to the sequencing technician to review quality. If the deviation is indeed confirmed (e.g., a novel variant is found), this causes an alert (e.g., a flag or message) to be sent to an agent (e.g., a computer module or a knowledge worker responsible for that gene. The module or worker can update the CORD™ Gene Annotation Database 160 is updated. For example, the module can evaluate the information and automatically update the database.

Each sequence can be appended to the GTO₂ (see the Gene Test Order process section) which then serves to populate the Person Variant database. The sequence variant is then matched against the CORD™ Gene Annotation Database 160. The corresponding Report(s) from Gene Report Database 150 (e.g., indexed by the same matching sequence variant) is then generated and forwarded as described in the Reporting Process 130.

Knowledge Discovery Process

CORD™ has an integral knowledge discovery process which uses as its inputs two databases:
1. The CORD™ Gene Annotation Database
2. The CORD™ anonymized Person Variant Database

The CORD™ anonymized Person Variant Database 174 has two data sources. The first is the standard DNA sequence and standard phenotypic annotations obtained during the Gene Test Ordering process. The second is a “phenotypic enrichment” data set that provides additional phenotypic data from third parties regarding persons whose DNA was sequenced through the CORD™ process. This includes, e.g., medical record companies, laboratory companies all of whom have important phenotypic characterizations of persons (e.g., laboratory values such as cholesterol, diagnosis codes, procedure codes). The demographic characteristics of the persons in these third party databases can be matched, e.g., probabilistically but highly accurately, against the same characteristics in the CORD™ Person Identification database 172, e.g., for some or all of persons in the CORD™ system. The matching process can produce phenotypic annotations of person-specific phenotypic annotation in order to improve the Knowledge Discovery Process 176.

In one embodiment, every time one of these two databases is updated, the CORD™ Knowledge Discovery Process (KDP). KDP software runs to update the probabilities linking all combination of data types in the CORD™ gene-variant-association model. This includes, e.g., gene variants to phenotypes, phenotypes to phenotypes, gene variants to gene variants

KDP assesses in a probabilistic framework (e.g., a Bayesian model or a comprehensive correlation structure) all the aforementioned dependencies. If any of these dependencies rises to the level of statistical significance, KDP first determines (based on the two databases) if the association is novel. If it is, KDP alerts an agent (e.g., a computer module or the knowledge worker ) regarding the new association. The agent assesses the association, e.g., to determine if it merits an update of the CORD™ Gene Annotation Database 160.

If KDP causes the CORD™ Gene Annotation Database 160 to be updated, then all persons with the relevant gene variant have updated reports generated as described in the CORD™ Gene Variant Annotation process 140. Reports can be sent, e.g., to a patient, general practitioner, billing agent, insurance company, specialist doctor, health care provider, or quality control agent.

Reporting Process

For each of the annotations in the Gene Annotation Database 160, the knowledge worker responsible for that gene will assign one of several clinical reports that are specific for a phenotypic association. These reports cover all contingencies from a high degree of confidence that the variant is casual of the phenotype to a high degree of confidence that it is not associated with the phenotype. Several intermediate levels of certainty and association are also reflected in the set of reports designed for a set of gene variants with respect to a phenotype.

The relationship between the report contents and the individual variants is maintained in the Gene Report database 150. There may be several report types for each gene variant to allow for different report content targeted for different readers and/or different purposes.

The reports can be forwarded to the ordering party or another party. Parties of interest include patient, general practitioner, billing agent, insurance company, specialist doctor, health care provider, or quality control agent.

Gene Test Ordering process

An ordered test consists of an order by a person whose sample will be tested or a third party acting on such person's behalf (e.g., the ordering agent) of either the analysis of a particular gene, a set of genes or the set of genes known to be associated with a phenotype/disease state. Each gene test order generates a Gene Test Order Object (GTO₂) that maintains a time-stamped and parse-able record in perpetuity of all aspects of the order. The outcome of the Gene Test Ordering process 110 is a set of reports for persons, providers and other parties authorized by the person, which describe the clinical implications of the variant(s) found for the person for whom the test was ordered.

To order a test, the ordering agent selects the gene, gene panel or phenotype for which they seek testing. Basic demographics to uniquely identify the person being tested are obtained but then are immediately escrowed into a separate database (Person Identifier database) and a unique semantic-free key is generated to link the GTO₂ to the person being tested. The ordering agent then supplies the required Minimum Phenotype Dataset (a small set of attributes) as well as an optional larger set of phenotypic attributes. The ordering agent also warrants, where required, that the person being tested has given an informed consent. The initial report can notify the recipient that if they sign and return an authorization that they may be contacted again after the first set of reports is generated if new knowledge is generated, e.g., information relevant to the health care of the person tested. The authorization is then cryptographically signed to authenticate its validity prior to its storage in the GTO₂.

Once the order is submitted, labels are generated for the containers of person tissue/blood, e.g., with the person's unique semantic-free key, and the tissue is obtained/blood and stored. A portion of the tissue/blood is used for DNA extraction and the DNA stored separately after a fraction of the DNA is sent to the DNA sequencer where the DNA is sequenced and the tracings of the sequencing output of the sequencer are submitted, along with the corresponding GTO₂, to the Sequence Interpretation Process 120.

Base Calling

An automated pattern recognition strategy, e.g., one which uses prior knowledge of the correct DNA sequence, would have advantages over an approach in which any nucleotide might appear at any position.

The pattern of nucleotide signals in known DNA sequence is used to compare with that of a test sequence. Two embodiments of pattern recognition include:
1) using a known DNA sequence (e.g., a sequence of the normal or wild-type gene) as the basis for comparison, and “training” the base calling program to a specific pattern, within a window of nucleotides of a given width, to acknowledge the importance of the immediate environment surrounding a given base to the appearance of that base in a chromatogram.
2) using a library of small (5-10 base) fragments of known DNA sequence (DNA fragment standards, DFS) which encompass many (e.g., 80, 90, 95%, or all) possible combinations, as the basis with which to read a test sequence. For example, if all possible combinations are used, and fragments of 5 nucleotides are used, the library would have 1024 DFS's. DFS's can be obtained, e.g., from pre-existing DNA sequences residing in DNA sequence repositories or generated de novo. For each unique DFS, the analysis of multiple examples is used to build a refined pattern, e.g., a pattern including or based on averages, and ranges, of sequence appearance.

In either case, the resulting reading of the test sequence can be used to further train the reading program for the interpretation of subsequent test sequences. For example, the sequence is modeled using a Markov approach.

Frequently the trace for a given nucleotide is influenced by the several (e.g., about four) bases that come before it. The trace can also be influenced by downstream bases within the template (e.g., the polymerase may “see” these downstream bases, or the higher order structure of the template downstream of the growing polymer may influence its growth).

The prediction method can account for sequencing rules, such as:
C's after T's are usually small
If there is more than one G after an A, the first G is small.
If there is more than one C after a G, the first C is small.
Sometimes in a string of 4 G's, the 2nd or 3rd G is small.
T's after G's are usually small.
In a string of 4 or more A's, the second A is usually small.

DFS's could be generated in plasmid vectors, and be sequenced. Alternatively, DNA sequence information in existing repositories, either diagnostic DNA sequencing centers or academic or commercial sequencing laboratories can be analyzed.

The size of the critical region used for DFS can be varied, e.g., to find a size which returns accurate reads, e.g., using a test set of sequence traces. The method can be used to generate patterns that are gene—and/or position-independent, e.g., with respect to terminal nucleotide appearance.

Patterns can generated by data mine a large repository of DNA sequence information to establish the correct pattern rules. The repository can employ the same DNA sequencing chemistry and DNA sequencing machines as will be used in future sequencing, as the patterns will likely be dependent upon both the chemistry and the machinery. In other words, patterns can be developed that are chemistry and/or machine specific. Other patterns may be general.

The patterns and rules can be used to evaluate (e.g., detect) the presence of heterozygous DNA bases at a given nucleotide position, by systematically introducing heterozygous nucleotides at each terminating position and analyzing the pattern. In one embodiment, Markov methods (e.g., hidden Markov models) are used for pattern recognition. In another embodiment, the program is trained, e.g., using a Bayesian model.

Computer Implementations

The invention can be implemented in digital electronic circuitry, or in computer hardware, firmware, software, or in combinations thereof. Methods of the invention can be implemented using a computer program product tangibly embodied in a machine-readable storage device for execution by a programmable processor; and method actions can be performed by a programmable processor executing a program of instructions to perform functions of the invention by operating on input data and generating output. For example, the invention can be implemented advantageously in one or more computer programs that are executable on a programmable system including at least one programmable processor coupled to receive data and instructions from, and to transmit data and instructions to, a data storage system, at least one input device, and at least one output device.

Each computer program can be implemented in a high-level procedural or object oriented programming language, or in assembly or machine language if desired; and in any case, the language can be a compiled or interpreted language. Suitable processors include, by way of example, both general and special purpose microprocessors. A processor can receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks. Any of the foregoing can be supplemented by, or incorporated in, ASICs (application-specific integrated circuits).

An example of one such type of system includes a processor, a random access memory (RAM), a program memory (for example, a writable read-only memory (ROM) such as a flash ROM), a hard drive controller, and an input/output (I/O) controller coupled by a processor (CPU) bus. The system can be preprogrammed, in ROM, for example, or it can be programmed (and reprogrammed) by loading a program from another source (for example, from a floppy disk, a CD-ROM, or another computer).

The hard drive controller is coupled to a hard disk suitable for storing executable computer programs, including programs embodying the present invention, and data including storage. The I/O controller is coupled by means of an I/O bus to an I/O interface. The I/O interface receives and transmits data in analog or digital form over communication links such as a serial link, local area network, wireless link, and parallel link.

One non-limiting example of an execution environment includes computers running Linux Red Hat OS, Windows NT 4.0 (Microsoft) or better or Solaris 2.6 or better (Sun Microsystems) operating systems. Browsers can be Microsoft Internet Explorer version 4.0 or greater or Netscape Navigator or Communicator version 4.0 or greater. Computers for databases and administration servers can include Windows NT 4.0 with a 400 MHz Pentium II (Intel) processor or equivalent using 256 MB memory and 9 GB SCSI drive. For example, a Solaris 2.6 Ultra 10 (400 Mhz) with 256 MB memory and 9 GB SCSI drive can be used. Other environments can also be used.

Other embodiments are within the following claims.

1. A method for diagnosing and periodically revising the level of confidence in the diagnosis of a cause of a disorder of a subject that presents with a phenotype associated with a disorder, the method comprising:
(1) providing a database of variants, the database comprising information about one or more variants associated with the disorder, and information associating each of the one or more variants with a level of confidence in the diagnosis of the disorder;
(2) determining the sequence of a target region of the gene in a subject, thereby providing sequence information for said subject;
(3) providing a first report for said subject that comprises information about the subject's sequence and the level of confidence in the diagnosis of the disorder, the report being determined by matching the subject's sequence information to one or more variants stored in the database, to thereby obtain information about the level of confidence in the diagnosis of the disorder given the subject's sequence information;
(4) modifying the database of variants; and
(5) providing a second or subsequent report for the subject, the second or subsequent report comprising information about the disorder as determined by comparing the subject's sequence information to one or more variants stored in the modified database, to thereby obtain information about the level of confidence in the diagnosis of the disorder.

2. The method of claim 1 wherein the sequence information used for providing the second or subsequent report is the sequence information obtained from the subject in conjunction with the issuance of the first report.

3. The method of claim 1 wherein the sequence information used for providing the second or subsequent report is obtained prior to generation of the first report.

4. The method of claim 1 wherein the physician uses the first, second or subsequent report to determine whether to deliver or withhold a selected treatment or to make a decision with regard to the management of the patient's care.

5. The method of claim 1 wherein the method is repeated for multiple subjects.

6. The method of claim 1 further comprising storing sequence and/or clinical information from the subject in a database that associates an identifier for each subject and the sequence and/or clinical information obtained from each subject.

7. The method of claim 1 wherein modifying the database of variants comprises altering at least one association between a variant and a disorder.

8. The method of claim 7 wherein altering at least one association comprises modifying the level of confidence in the diagnosis of the disorder.

9. The method of claim 1 wherein modifying the database of variants comprises adding at least one association between a variant and a disorder.

10. The method of claim 9 wherein adding at least one association comprises modifying the level of confidence in the diagnosis of the disorder.

11. The method of claim 1 wherein modifying the database of variants comprises adding a new variant that was absent from the database prior to the modifying.

12. The method of claim 1 wherein providing a modified database of variants comprises determining the sequence of the target region of the gene in a second or subsequent subject; and modifying the database of variants based on information about the second subject or any subsequent subject.

13. The method of claim 12 wherein the subsequent subject is not a subject who has been previously tested and to whom a first report has not yet been issued.

14. The method of claim 1 wherein modifying the database of variants comprises evaluating new associations.

15. The method of claim 1 wherein at least one of the reports comprises the interpretation of the results of the subject's sequence information, the subsequent reports are provided as warranted by subsequent changes in the database of variants.

16. The method of claim 15 wherein the changes in the database of variants comprise changes that alter the level of confidence between the subject's sequence information and the diagnosis of the disorder.

17. The method of claim 1 wherein the variants comprise single nucleotide polymorphisms.

18. The method of claim 1 wherein the variants comprise one or more of a deletion of at least one nucleotide, an inversion, a translocation, or an insertion of at least one nucleotide.

19. The method of claim 1 further comprising, prior to determining the sequence of a target region of the gene in the test subject, receiving (i) a requisition that requests sequence information for the subject and/or (ii) clinical information about the test subject.

20. The method of claim 1 wherein the second or subsequent report includes information about the level of confidence in the diagnosis of the disorder.

21. The method of claim 20 wherein the level of confidence in the second or subsequent report is revised relative to a previous report.

22. The method of claim 20 wherein the second report or subsequent report indicates a different level of confidence in the diagnosis of the disorder than that indicated in a corresponding first or previous report.

23. The method of claim 20 wherein the second or subsequent report indicates that the level of confidence in the diagnosis is unchanged compared with the first or previous report.

24. The method of claim 1 wherein the first and second report are one or a series of at least three reports.

25. The method of claim 1 wherein identifying variants comprises a step of comparing the sequence information for a subject to a reference sequence.

26. The method of claim 1 further comprising storing, for each of the first subjects, an indicator that represents whether a subject requests an updated report for his/her genetic information.

27. The method of claim 1 further comprising requesting and/or receiving additional clinical information for one or more of the subjects.

28. The method of claim 1 wherein the database of variants comprises one or more database entries that correlate a combination of variants and a clinical state.

29. The method of claim 1 wherein the report further comprises information about state of the database.

30. The method of claim 1 wherein the step of preparing a subsequent report comprises:
detecting changes to the table of variants;
accessing a database that comprises sequence information for multiple individuals; and
identifying individuals that require a subsequent report.

31. The method of claim 1 further comprising receiving a request for testing.

32. A method comprising:
preparing a first report that provides a diagnosis for a disorder based on sequence information about a first subject, the sequence information including information about a gene;
storing the sequence information about the subject;
updating a system that stores information about variants in the gene with data external to said system;
determining if a change in the system of variants alters the diagnosis for the disorder as reported for the subject in the first report; and
optionally, preparing a subsequent report for the subject that provides a diagnosis for the disorder based on evaluating the subject's sequence information using the updated system.

33. The method of claim 32 wherein the data that is used to update the system is acquired from other test subjects and/or from new knowledge from scientific literature or other sources.

34. The method of claim 32 wherein the second or subsequent report is prepared if the level of confidence in the diagnosis is altered.

35. The method of claim 32 wherein the subsequent report is prepared whether or not the level of confidence is altered and the subsequent report includes information that the level of confidence in the diagnosis is unchanged in the case where no alteration is detected.

36. The method of claim 32 wherein the table of variants comprises references that link a particular variant to stored sequence or clinical information about subjects that have the particular variant.

37. The method of claim 32 wherein clinical information or the sequence information about each subject is stored in a database.

38. The method of claim 37 further comprising monitoring one or more of the subjects for a clinical parameter.

39. The method of claim 37 further comprising requesting and/or receiving information from physician or subject.

40. The method of claim 39 wherein the request or receipt is made if the subject has a variant that has not been correlated with the disorder at the time of the first report.

41. A system comprising
a database of sequence information that associates identifiers for individuals and sequence information for one or more genes that are associated with a disorder;
a database of variants that associates variants in the one or more genes and the disorder;
one or more processors, configured to access each of the databases and execute a method comprising:
(i) receiving sequence information and clinical information for a subject;
(ii) appending, to the database of sequence information, a record that associates an identifier for the subject and the received sequence information;
(iii) identifying one or more variants in the received sequence information;
(iv) if the identified variant(s) is present in the database, retrieving an indication of the level of confidence that the variant is associated with the disorder from the database of variants and generating a report that comprises the retrieved information; and
(v) determining, from the sequence information and the clinical information for the subject, if the database of variants requires modification.

42. A method comprising:
assessing a database or an online-index of biomedical information to identify information about a gene that is new relative to a previous assessment;
evaluating the new information using stringency criteria; generating a test rule based on the new information; and
processing a database of information in which records for individuals associate genetic information to phenotypic information using the test rule.

43. The method of claim 42 wherein the assessing is effected periodically.

44. A method for diagnosing and reporting a disorder, the method comprising:
providing a database of variants, the database comprising associations between one or more variants, and the disorder, wherein at least one of the associations comprises a characterization of quality of the associations;
determining the sequence of a target region of the gene in a subject, thereby providing sequence information for multiple subjects; and
providing a report for each subject that comprises information about the subject's sequence and the level of confidence in the diagnosis of the disorder as determined by comparing the subject's sequence information to information about associated levels of confidence annotated in the database of variants.

45. A method for diagnosing and reporting a diagnosis of a disorder, the method comprising:
evaluating a study that provides an association between a variant and a disorder to obtain a qualitative or quantitative indicator of quality for the association;
modifying a database of variants such that the database stores the association and the indicator of quality;
determining the sequence of a target region of the gene in a subject, thereby providing sequence information for multiple subjects; and
providing a report for each subject that comprises information about the subject's sequence and the level of confidence in the diagnosis of the disorder as determined by comparing the subject's sequence information to information about associated levels of confidence annotated in the database of variants.

46. The method of claim 45 wherein the indicator of quality is based on a linear weighting of quality of the study.

47. The method of claim 45 wherein the indicator of quality is:
a parameter indicating the quality of phenotypic-genotypic association based on the knowledge of the pedigree and/or association studies used to populate the database, or an estimate thereof;
a parameter indicating the quality of functional studies performed by one or more researchers to determine the functional significance of a particular variant, or an estimate thereof; or
a parameter indicating the likelihood that a given variant will cause a change in function and/or phenotype based on the nature of the change of the coded amino acid, the change of a conserved sequence, the chance of an important part of a functional domain of a gene/protein, or an estimate thereof.

48. The method of claim 45 wherein the indicator of quality is based on a linear weighting of two or more of the following parameters:
a parameter indicating the quality of phenotypic-genotypic association based on the knowledge of the pedigree and/or association studies used to populate the database, or an estimate thereof;
a parameter indicating the quality of functional studies performed by one or more researchers to determine the functional significance of a particular variant, or an estimate thereof; and
a parameter indicating the likelihood that a given variant will cause a change in function and/or phenotype based on the nature of the change of the coded amino acid, the change of a conserved sequence, the chance of an important part of a functional domain of a gene/protein, or an estimate thereof.
